(19) United States
(12) Patent Application Publication
(10) Pub. No.: US 2015/0018717 A1
(43) Pub. Date: Jan. 15, 2015

(54) APPARATUS AND METHOD FOR ACCURATE CUN SELF MEASUREMENTS

(71) Applicant: Kevin P. Lyons, Charlotte, NC (US)

(72) Inventor: Kevin P. Lyons, Charlotte, NC (US)

(21) Appl. No.: 14/325,623

(22) Filed: Jul. 8, 2014

Related U.S. Application Data
(60) Provisional application No. 61/844,621, filed on Jul. 10, 2013.

(51) Int. Cl. A61B 5/00; A61H 39/02

(52) U.S. Cl. CPC A61B 5/4854; A61H 39/02
USPC 600/587

(57) ABSTRACT

An apparatus and method for self calibration of ‘cun’ measurements comprising, a rubber tubing or other tubing with elastic properties, a set of concave shaped beads fixed on the two ends of the rubber tubing to act as hand grips, a set of four colored beads that move freely along the rubber tubing, placing the widest portion of the thumb between the blue and green colored beads to calibrate the 1 ‘cun’ measurement, placing the index finger and the middle finger between the green and yellow beads to calibrate the 1.5 ‘cun’ measurement and placing all four fingers between the yellow and orange beads to calibrate the 3 ‘cun’ measurement. The self calibrated apparatus can be placed on any part of the body to locate acupuncture points for treatment with acupuncture needles, patches, or other forms of stimulation such as heat therapy, laser therapy, acupressure and electrical stimulation.

### CROSS REFERENCE TO RELATED APPLICATION

This application claims priority under 35 USC §119 (e) to U.S. Provisional Patent Application, Ser. No. 61/844,621 filed Jul. 10, 2013 which is incorporated herein in its entirety.

### FIELD OF THE INVENTION

This invention is generally related to acupuncture techniques and devices. More particularly, the invention relates to an apparatus and method for accurately measuring the ‘cun’ (human inch) to locate acupuncture points on the body when administering self-acupuncture.

### BACKGROUND OF THE INVENTION

Acupuncture is a healing technique that has its roots of origin in China. It has been an important component of Traditional Chinese Medicine (TCM) for centuries. In the past decades, the beneficial effects of acupuncture in alleviating a variety of conditions has been recognized and accepted by the Western world.

The techniques used in the practice of Acupuncture are aimed at correcting imbalances in the flow of energy through the body. This energy is referred to as the ‘qi’ in TCM. In acupuncture, balancing the flow of ‘qi’ is accomplished by stimulating specific locations on the body called acupuncture points or acupoints using thin sterilized needles inserted through the skin surface and manipulating the needles or applying other stimulation to these acupoints such as acupuncture patches.

Acupuncture points or acupoints are generally located along ‘meridians’ in different parts of the human body through which the energy ‘qi’ flows. Meridians exist in corresponding pairs in the human body and each meridian has many acupuncture points along its path. The location of the meridians and the corresponding acupuncture points are described widely in the literature. Of particular note, is the text book, “Essentials of Chinese Acupuncture”, written and compiled by the Beijing College of Traditional Chinese Medicine, published in 1980 by the Foreign Languages Press, Beijing. According to the book and other acupuncture literature, there are generally 14 meridians in the acupuncture system of traditional Chinese medicine. These meridians are identified as, 1.Conception Vessel, 2.Gall Bladder, 3.Governing Vessel, 4.Pericardium, 5.Bladder, 6.Triple Warmer, 7. Lung, 8.Heart, 9.Kidney, 10.Small Intestine, 11.Large Intestine, 12.Liver, 13.Spleen and 14.Stomach. The acupuncture points or acupoints as they are generally referred to, run along these meridians and are numbered consecutively, starting at the tips of the toes and the fingers, except for the Conception Vessel meridian (starting at the front lower midline portion of the body) and the Governing Vessel meridian (starting at the head and runs midline down the back of the body. Meridian therapy is the accepted name employed by those who practice acupuncture with or without the use of penetrating needles. Other forms of stimulation such as electric stimulation, laser therapy, acupressure and heat therapy are also used to treat the acupoints.

Correctly locating acupuncture points on the body is critical for the treatment of various disease conditions using the healing method of acupuncture. In order to accurately locate the acupuncture points on the human body, the first step is to determine the patient ‘cun’ (pronounced, tsōōn) also known as the ‘Chinese inch’ or “human inch” which is a variable unit of length. Measuring the ‘cun’ is considered the best way to locate acupoints on the body. Generally speaking, one ‘cun’ is equal to the widest portion of the patient's thumb and/or the space between the distal inter-phalangeal joint (the joint between the second and third finger bones) and the proximal inter-phalangeal (the joint between the first and second finger bones) on the middle finger. The ‘cun’ measurements are unique to each individual's body type.

Prior art for taking ‘cun’ measurements to locate acupuncture points on the body have focused on devices that can be used and operated only by an acupuncture physician. Some of these comprise, electronic acupuncture point locators that operate by passing an electrical current through the body to locate the acupoints as described in the acupuncture point position evaluating apparatus of U.S. Pat. No. 7,818,054 (Fukumoto et al). Other prior art require the use of electronic acupuncture sensor equipment. Yet other prior art use 3D scanning of the curved surface of a human body to determine the acupuncture points as described in U.S. Patent. Appl. Pub. No. 2013/0027368 (Lee et al.).

Prior art that are meant to be used to practice self acupuncture, have proven to be not very effective, due to the user's inability to locate the acupoints correctly using those procedures and devices. U.S. Pat. No. 4,520,798 to Lewis describes a self acupressure method and apparatus comprising a plate vertically mounted on a wall with arms connected to the plate having bars and knobs, placing the body area to be treated in contact with the knobs and applying intermittent pressure to that body area. This apparatus and method appears to be a cumbersome means for self acupressure treatment. Some self acupuncture procedures in the prior art require the use of an electronic device to calibrate the patient's ‘cum.’ Prior art do not provide any instrument, apparatus or method that is non-electronic to calibrate a patient's ‘cun.’ The present invention provides such a device and method to accurately calibrate a patient's ‘cun’ and locate the acupuncture points or acupoints correctly by a patient on their own without the use of electronic equipment or other weighty and cumbersome devices, or the assistance of a trained acupuncture professional.

### SUMMARY OF THE INVENTION

The present invention is an apparatus and method that assists a patient in accurately locating the acupuncture points on their bodies by themselves based on anatomical landmarks without the use of electronic acupuncture sensor equipment, nor assistance from their acupuncture physicians or healers in order to administer stimulation at those acupuncture points to treat their condition. The apparatus of the present invention is compact, light weight and easily portable.

In an exemplary embodiment of the present invention, the apparatus is a device and system designed for the users to calibrate the device into distinct numerical ‘cun’ (human inch/Chinese inch) units for the purpose of locating acupuncture points on their own bodies with the use of guided mapping coordinates, thereby enabling the patient to locate and stimulate acupuncture points on themselves. In this embodiment of the invention, the calibration of the apparatus is accomplished by measuring at least three ‘cun’ to locate acupuncture points along a meridian on specific parts of the body. For purposes of calibration of the apparatus of the present invention, generally speaking, 1 ‘cun’ is the widest portion of the first phalange (finger bone) at the inter-phalange (joint between the finger bones) point, 1.5 ‘cun’ is the width between the second and third distal phalange together, and 3 ‘cun’ is the width between phalanges one through four measured together. The method for self calibration is illustrated in a guide provided to the patient by the acupuncture practitioner.

In the preferred embodiment of the invention, the apparatus consists of a thick hollow rubber tubing 2 millimeters (mm) in width and 10 inches in length that has static and elastic properties. There are two 30 mm concave shaped plastic beads fixed on the rubber tubing to be used as grips and an additional four plastic 8 mm colored beads used for marking the distance between the ‘cun’ measurement anatomical points. The four colored beads are capable of being moved along the rubber tubing between the fixed beads. In this embodiment, when the two elastic ends of the rubber tubing are stretched, the four plastic colored beads move freely along the rubber tubing allowing for easy personal patient calibration of the device. When the rubber tubing ends are released after calibration, the colored beads remain stationary and fixed due to the increase in girth and friction of the tubing upon release.

In the exemplary embodiment of the invention, the patient initially calibrates the apparatus to three ‘cun’ measurements, particularly, 1 ‘cun’, 1.5 ‘cun’, and 3 ‘cun.’ This is accomplished by first extending the rubber tubing of the apparatus holding the colored beads. In order to determine the measurement for 1 ‘cun’ the widest portion of the thumb is placed between the blue and green beads. Once the 1 ‘cun’ measurement is taken and the blue and green beads are stabilized and held in place on the rubber tubing, the index and middle finger are placed between the green and yellow beads to provide the 1.5 ‘cun’ measurement. The next step is to take the 3 ‘cun’ measurement by stretching the rubber tubing and placing the index, middle, ring and pinky fingers together on the tubing between the yellow and the orange beads. Once the apparatus is thus calibrated, it can be placed on a body surface such as for example the inner surface of an extended hand and the ‘cun’ measurements calibrated on the apparatus can be used to locate the acupuncture points for treatment using needles, patches, or other forms of stimulation such as heat therapy, laser therapy, acupressure and electrical stimulation.

There are about 359 standard acupuncture points on the human body. Each acupuncture point can be located using an anatomical landmark starting point and one or a combination of the ‘cun’ measurements. Using the embodiments of the ‘cun’ self measuring apparatus of the present invention, patients can more accurately measure and find acupuncture points on their bodies on their own using anatomical landmarks as starting points.

All embodiments of the ‘cun’ self measurement apparatus of the present invention, are compact, light weight and easily portable with an accompanying illustrated guide in the form of a map, with clear, easy to comprehend, instructions and designed to be functional on a mathematical tactile and/or visual basis across all languages as well as for use by the visual and hearing impaired. The apparatus does not require the need to be plugged into an external power source or the use of batteries for its operation. The elastic properties of the device enable the user to use just one hand to hold the apparatus for calibration. The apparatus of the invention can be constructed with the use of any suitable material besides rubber that has elastic properties. Various other designs besides the one described in this specification are envisioned for the apparatus of the present invention. The apparatus of the present invention has been tested on volunteers who were able to accurately locate acupuncture points on their bodies using the calibrated apparatus and the results verified using an electronic computer based point locator.

The foregoing description and summary of the ‘cun’ self measurement apparatus of the present invention through its preferred embodiments should not be construed to limit the scope of the invention. It should be understood and obvious to one skilled in the art that the embodiments of the invention thus described may be further modified without departing from the spirit and scope of the invention.

### BRIEF DESCRIPTION OF THE DRAWINGS

FIG. 1 is a perspective view of the front, rear and side of the human body illustrating the known meridians used in the practice of acupuncture.

FIG. 2 is a perspective view of the exemplary embodiment of the ‘cun’ self measurement apparatus of the present invention.

FIG. 3 is a perspective view of the first step in the calibration of the apparatus which is to determine the 1 ‘cun.’

FIG. 4 is a perspective view of the second step in the calibration of the apparatus which is to determine the 1.5 ‘cun.’

FIG. 5 is a perspective view of the third step in the calibration of the apparatus which is to determine the 3 ‘cun.’

### DETAILED DESCRIPTION OF THE INVENTION

The present invention is an apparatus and method for accurately locating and measuring the acupuncture or acupoints on a person's body without the use of electrical, sensory, 3D, or other cumbersome and weighty equipments or devices. The compact, light weight and portable apparatus of the invention, allows the patient or user to self measure their acupoints in the privacy of their own homes and treat themselves through the principles of acupuncture. The user of the apparatus of the invention follows a guided map to calibrate the device into distinct numerical ‘cun’ units to locate acupuncture points on their bodies and stimulate the acupoints using needles, patches, or other forms of stimulation such as heat therapy, laser therapy, acupressure and electrical stimulation to treat their maladies or conditions on their own.

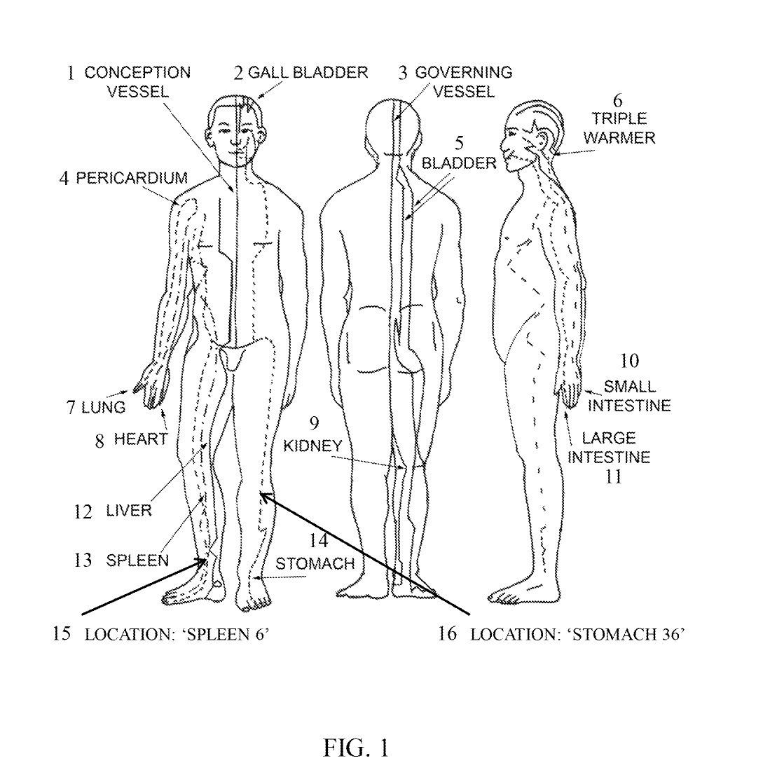

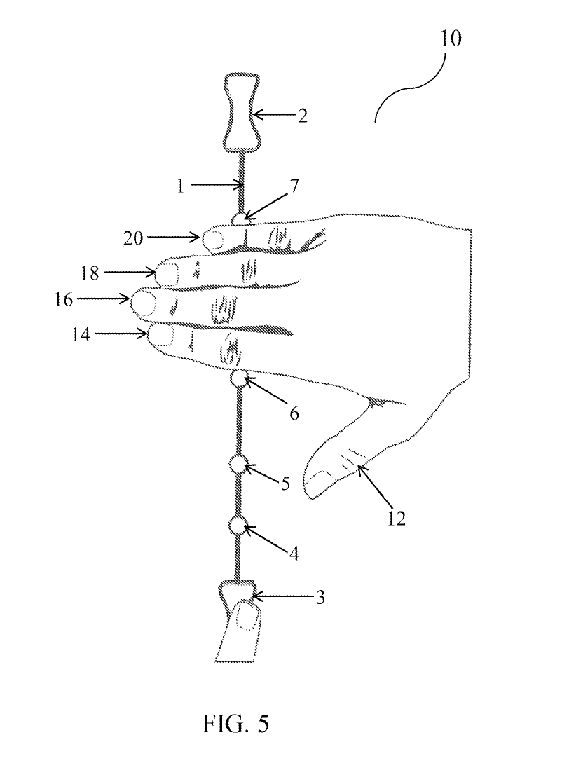

Referring now to the figures, FIG. 1 shows a perspective view of the front, rear and side of the human body illustrating the locations of the meridians used in acupuncture practice to locate the acupuncture points or acupoints on the body for treatment using needles, patches or by other means of stimulation. This illustration is taken from FIG. 5, page 86 of the book “Essentials of Chinese Acupuncture” written and compiled by the Beijing College of Traditional Chinese Medicine, published in 1980 by the Foreign Languages Press, Beijing.

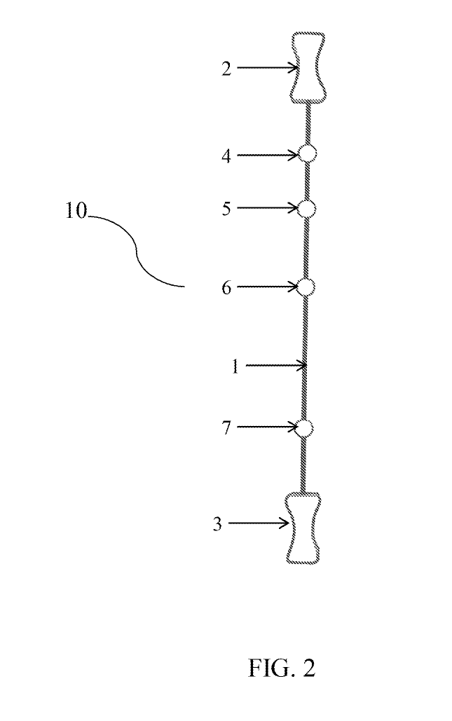

FIG. 2 is a perspective view of the preferred and exemplary embodiment of apparatus 10 of the present invention which is calibrated to locate acupuncture or acupoints on the human body for treatment using meridian therapy in the practice of acupuncture. Apparatus 10 is comprised of a thick, hollow rubber tubing 1 approximately 2 millimeters (mm) in width and 10 inches in length that has static and elastic properties. Two large concave shaped beads 2 and 3 constructed from plastic or other suitable material are fixed one to the first end and the other to the second of the rubber tubing 1 and are used as grips for extending and holding the rubber tubing 1 in place with one hand A set of colored beads, blue 4, green 5, yellow 6 and orange 7 also constructed of plastic or other suitable material are held on the rubber tubing 1 in that order between the fixed concave shaped beads 2 and 3. The colored beads, 4, 5, 6 and 7 are capable of movement along the length of the rubber tubing 1 between the fixed concave shaped beads 2 and 3. In this embodiment, when the two ends of the rubber tubing 1 are stretched, the four colored beads, 4, 5, 6 and 7 move freely along the rubber tubing 1 allowing for easy personal patient calibration of the device. When the rubber tubing 1 ends comprising the concave shaped beads 2 and 3 are released after calibration, the colored beads, 4, 5, 6 and 7 remain stationary at their positions after calibration on the rubber tubing 1 due to the increase in girth and the friction of the rubber tubing 1 upon its release.

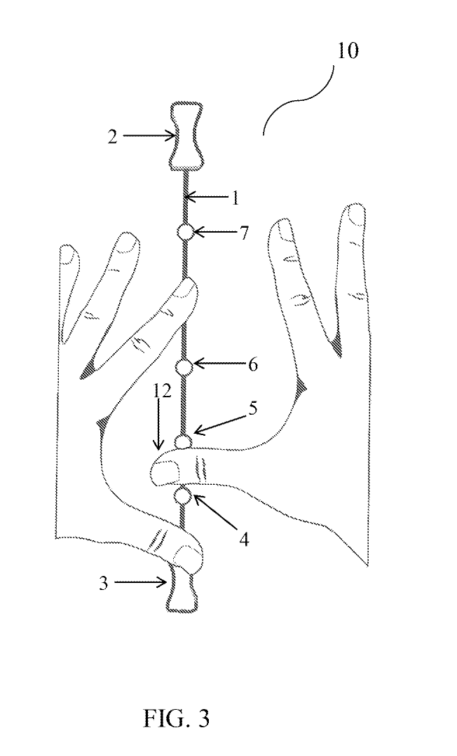

In the exemplary embodiment of the invention, the patient initially calibrates the apparatus 10 to three ‘cun’ measurements, particularly, 1 ‘cun’, 1.5 ‘cun’, and 3 ‘cun.’ This is accomplished by first extending the rubber tubing 1 of the apparatus holding the colored beads 4, 5, 6 and 7 by gripping the concave shaped beads 2 and 3 and pulling the rubber tubing 1 to either side and holding the rubber tubing 1 in this stretched position with one hand. In order to determine the measurement for 1 ‘cun’ the widest portion of the thumb 12 on the other hand is placed between the blue bead 4 and the green bead 5. This first step of calibrating the apparatus 10 to the 1 ‘cun’ measurement is illustrated in FIG. 3. Once the 1 ‘cun’ measurement is thus calibrated and noted, the thumb 12 is removed from between the blue bead 4 and the green bead 5 and these two beads are stabilized and held in place while moving to the second step of calibration.

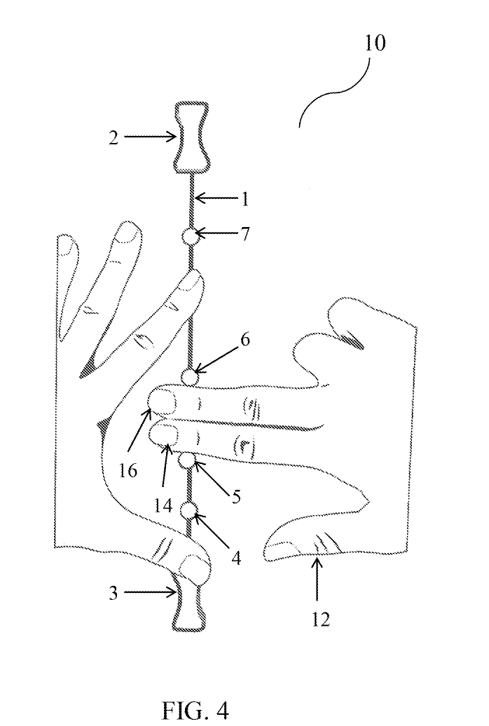

FIG. 4 illustrates the second step in the calibration of the apparatus 10 to the 1.5 ‘cun’ measurement. In this step as in the first step, the rubber tubing 1 is first extended using the concave shaped beads 2 and 3 at the ends of the rubber tubing 1 and held in place with the same hand used in the first ‘cun’ calibration. Thereafter, the index finger 14 and the middle finger 16 of the other hand are placed between the green bead 5 and the yellow bead 6 to provide the 1.5 ‘cun’ measurement.

FIG. 5 illustrates the third and last step in the calibration process of the apparatus 10. In this step, after the rubber tubing 1 is extended using the same process as described for the first and second step, all four fingers, the index finger 14, the middle finger 16, the ring finger 18 and the pinky finger 20 are placed together side by side between the yellow bead 6 and the orange bead 7 which provides the 3 ‘cun’ measurement.

Once the apparatus 10 is calibrated to the three ‘cun’ measurements, it can be placed on the surface of a body at an anatomical landmark such as for example the distal wrist crease on the inner surface of an extended hand and wrist. The ‘cun’ measurements calibrated on the apparatus 10 can then be used to locate the acupuncture points on the forearm given the selected points coordinates from the land mark, for treatment using needles or other forms of stimulation such as heat therapy, laser therapy, acupressure and electrical stimulation. The self calibrated apparatus 10 can be placed on any part of the body to locate any and all acupuncture points on the body.

Using the embodiments of the ‘cun’ self measuring apparatus of the present invention, patients can more accurately measure and find acupuncture points on their bodies using anatomical landmarks as starting points. For example, if a patient wanted to locate the ‘Spleen 6’ acupuncture point located 3 cun above the apex of the medial malleolus (spherical bony protrusion on the inner side of the leg just above the ankle joint) behind the tibia (shinbone), they would first find the highest point of the medial malleolus and then using the calibrated apparatus, measure 3 ‘cun’ cephalad (towards the head end) of the inner knee. At the point where the 3 ‘cun’ measurement terminates lays the acupuncture point for ‘Spleen 6’. In another example, if a patient wanted to locate the ‘Stomach 36’ acupuncture point, located 3 cun below the tibial tuberosity (spherical bony protrusion on the upper shinbone below the knee cap) of the tibia on the lateral aspect of the tibialis anterior muscle (the muscle located directly on the shinbone) they would measure 3 cun caudad (towards the tail end, yellow to orange bead) directly below the bottom edge of the patella (knee cap) and 1.5 cun (green to yellow bead) lateral toward the outside of the leg, away from the body. This would be the location of the ‘Stomach 36’ acupuncture point. The apparatus of the present invention not only allows for self calibration to calculate a variety of ‘cun’ measurements, but also assists the patient or user to locate any and all of their acupuncture points on their own to treat themselves using the acupuncture technique.

The foregoing detailed description of the present invention through its figures and exemplary embodiments should not be construed to limit the scope of the invention. It should be understood that the phraseology and terminology used to describe the various parts of the invention are for descriptive purposes only and other phrases and terminology may be used to describe the relevant parts of the invention without departing from the scope of the invention. It should be further understood and obvious to those skilled in the art that alternatives, modifications and variations of the embodiments of the invention described herein are within the spirit and scope of the appended claims.

What is claimed is:

1. An apparatus for self calibration of ‘cun’ measurements to locate acupuncture points the apparatus comprising:
a thick hollow rubber tubing with elastic properties having a first end and a second end;
two concave shaped beads one fixed to said first end and the other fixed to said second end of said rubber tubing to act as hand grips;
four colored beads, blue, green, yellow and orange in that order held on said rubber tubing between the said concave shaped bead on said first end and said concave shaped bead on said second end of said rubber tubing; and
said four colored beads capable of moving freely along the length of said rubber tubing.

2. The apparatus of claim 1 wherein the ‘cun’ measurements are calibrated using the colored beads and the finger's of a user's hand.

3. The apparatus of claim 1 wherein measurement for 1 ‘cun’ is taken by extending the rubber tubing using the concave shaped beads fixed to the two ends of the tubing and placing the widest portion of the thumb between the blue bead and the green bead.

4. The apparatus of claim 1 wherein measurement for 1.5 ‘cun’ is taken by extending the rubber tubing using the concave shaped beads fixed to the two ends of the tubing and placing the index finger and the middle finger between the green bead and the yellow bead.

5. The apparatus of claim 1 wherein the measurement for 3 ‘cun’ is taken by extending the rubber tubing using the concave shaped beads fixed to the two ends of the tubing and placing the index finger, middle finger, ring finger and the pinky finger between the yellow bead and the orange bead.

6. The apparatus of claim 1 wherein the apparatus when self calibrated can be used to locate any acupuncture points on the human body for treatment with acupuncture needles, patches, or other forms of stimulation such as heat therapy, laser therapy, acupressure and electrical stimulation.

7. The apparatus of claim 1 wherein the beads of the apparatus are constructed from plastic or other suitable material.

8. The apparatus of claim 1 wherein the colored beads can be of any color.

9. The apparatus of claim 1 wherein the apparatus of the invention can be constructed with the use of any suitable material besides rubber that has elastic properties.

10. A method of locating acupuncture points on the human body on one's own without the assistance of an acupuncture practitioner, the method comprising:
a) providing an apparatus for self calibration of ‘cun’ measurements comprising:
a rubber tubing with concave shaped beads fixed to both ends of the rubber tubing to serve as hand grips;
a set of colored beads, blue, green, yellow and orange in that order held on the rubber tubing between the fixed concave shaped beads;
b) extending the rubber tubing with the concave shaped beads with one hand;
c) placing the widest portion of the thumb of the other hand between the blue bead and the green bead, to calculate the 1 ‘cun’ measurement;
d) placing the index finger and the middle finger of the same hand between the green bead and the yellow bead, to calculate the 1.5 ‘cun’ measurement; and
e) placing the index finger, middle finger, ring finger and the pinky finger of the same hand between the yellow bead and the orange bead, to calculate the 3 ‘cun’ measurement.

11. The method of claim 10 wherein after the apparatus is self calibrated for the ‘cun’ measurements, the apparatus is placed on any part of the body to locate the acupuncture points to place the acupuncture needles, patches, or other forms of stimulation such as heat therapy, laser therapy, acupressure and electrical stimulation.
